(19) United States
(12) Patent Application Publication
(10) Pub. No.: US 2012/0097155 A1
(43) Pub. Date: Apr. 26, 2012

(54) SYSTEM AND METHOD FOR INTEGRATED PACED BREATHING AND INHALATION THERAPY

(75) Inventor: Vijay Kumar Iyer, Export, PA (US)

(73) Assignee: KONINKLIJKE PHILIPS ELECTRONICS N.V., EINDHOVEN (NL)

(21) Appl. No.: 13/377,217

(22) PCT Filed: Jun. 16, 2010
(86) PCT No.: PCT/IB2010/052714

§ 371 Date: Dec. 9, 2011

Related U.S. Application Data
(60) Provisional application No. 61/224,118, filed on Jul. 9, 2009.

(51) Int. Cl. A61M 16/10; A61M 15/00; A61M 11/00; A61M 16/18; A61M 16/12; A61M 16/00; A61M 16/06

(52) U.S. Cl. 128/200.14; 128/203.12; 128/203.14; 128/203.15; 128/203.29

(57) ABSTRACT

The invention provides systems and methods for providing integrated paced breathing and inhalation therapy to a patient. Paced breathing includes alternately providing a high and a low airway pressure to a respiratory system of the patient using a breathing circuit, wherein the high airway pressure coincides with inhalation by the patient and the low airway pressure coincides with exhalation by the patient. A therapeutic substance is delivered to the respiratory system of the patient during the provision of high airway pressure to the respiratory system of the patient, wherein delivery of the compound to the respiratory system of the patient is discontinued during provision of low airway pressure to the respiratory system of the patient.

The invention relates to systems and methods for integrated paced breathing and inhalation therapy.

Aerosolized drugs/therapeutics are often used in conjunction with a nebulizer or a metered dose inhaler. In some instances, aerosolized drugs are delivered with invasive ventilation in the hospital setting for administering drugs directly to the lung for the treatment of lung diseases.

Paced breathing therapy was developed to address, among other things, the needs of patients needing to reduce their sympathetic nervous system arousal and activity. This activity may include hypertension, insomnia, or other ailments. In some instances, patients with these ailments are expected to use drugs as conjunctive therapy for their disease. In some instances, patients with these ailments also have co-morbid conditions along with their primary disease such as, for example, hypertension with diabetes, hypertension with conjunctive heart failure (CHF), hypertension with asthma, insomnia with hypertension, chronic obstructive pulmonary disease (COPD) with asthma. In some instances, more than two co-morbid conditions may exist. As such, the administration of integrated therapies including paced breathing and an inhaled compound maybe valuable. For example, some of the patients may need a relaxant to help them to use paced breathing therapy. This may be provided in the form of humidification, aromatherapy, a relaxant drug, and/or other inhaled therapy.

While paced breathing serves to reduce sympathetic nervous system arousal/activity, inhaled compounds may also be used to help these patients relax and to reduce sympathetic system activity. Other compounds/drugs may be employed that address other issues like their hypertension, CHF, diabetes, Asthma, COPD, insomnia, etc. with humidification, diuretics, ACE inhibitors, ARBS, insulin, bronchodilators or corticosteroids, antibiotics, sleep inducers, etc. This can enhance the overall efficiency of treatment administration to the patient and may improve adherence to the therapies.

As such, systems and methods that effectively combine paced breathing and inhalation therapy may be desirable.

As paced breathing therapy provides a controlled (typically slower) breathing rate, deeper breathing, a more regular/stable breathing rate, a controlled inhalation/exhalation ratio, and/or other control over patient breathing, it is advantageous to utilize paced breathing in conjunction with conducive the administration of inhaled compounds to a patient. As such, the invention provides systems and methods for integrated ventilatory paced breathing and inhaled substance therapy.

A system for combined ventilatory paced breathing and inhaled substance therapy may include a breathing circuit, a drug delivery component, and a control component. The breathing circuit may include a ventilator or other pressure manipulation device, a breathing conduit, and a patient interface (e.g., a mask, nasal cannula, or other interface). The breathing circuit may be configured to selectively provide different airway pressures to the respiratory system of a patient and/or otherwise provide airflow or breathing regulation or assistance to the respiratory system of the patient.

In some implementations, the breathing circuit may enable control of a patient's breathing rate, including slowing down or speeding up the breathing rate from what it would be without the use of paced breathing therapy. In some implementations, the breathing circuit may enable control of breath volume (i.e., tidal volume) of the patient, including increasing or decreasing the breath volume from what it would be without the use of paced breathing therapy. In some implementations, the breathing circuit may enable control of the regularity of the patient's breathing. In some implementations, the breathing circuit may enable control of the ratio between inhalation duration and exhalation duration (“I/E ratio”).

The drug delivery component may vary depending on the type of compound being delivered to the patient. For example, to administer an aerosolized, powder or liquid, the drug delivery component may include a nebulizer as a selective drug delivery portion of drug delivery component. To administer a gaseous therapeutic substance, the drug delivery component may include a compound source (e.g., a tank of pressurized gas containing the compound to be delivered [e.g., oxygen or other gas]), a conduit to deliver the gas from the source to the breathing circuit, and a valve as a selective drug delivery portion to selectively allow flow from the source through the conduit to the breathing circuit and/or the respiratory system of the patient.

The control component may be or include a control unit which may be or include a computer-implemented device having one or more computer processors (e.g., microprocessors) and associated memory or data storage. In some implementations, the one or more processors may be configured to perform one or more operations relating to paced breathing and inhalation therapy described herein. The control unit may include and the one or more processors may be configured by a control application.

The control application may include and/or be programmed with a paced breathing algorithm so as to provide various airway pressures to the respiratory system of the patient for treatment of one or more medical conditions. In some implementations, paced breathing therapy employs positive air pressure (PAP) cues as a more natural proprioceptive signal to coach patients to breathe at a slow, therapeutically significant breath rate (e.g., <10 breaths per minute (BPM)). This may provide several physiological benefits such as, for example, reduction of blood pressure and/or other benefits. As discussed herein, paced breathing may be used to control breathing rate, tidal volume, regularity of breathing, I/E ratio and/or other characteristics of a patient's respiration. The paced breathing algorithm used to control airway pressure/duration may vary according to the desired characteristics of patient respiration.

The paced breathing algorithm may be implemented using one or more breathing signals from the control unit to the ventilator that instruct ventilator to create a specific level of positive pressure in the breathing circuit or otherwise cause flow within the breathing circuit. In some implementations, the breathing signal may also be used to control the timing of drug/therapeutic substance delivery to optimize therapy delivery and economize on the use of expensive compounds. In some instances, control of therapeutic substance delivery based on a paced breathing signal may increase the effectiveness of inhalation therapy by significantly enhancing the transport of inhaled substances to the lung.

For example, in some implementations utilizing the breathing circuit to induce a slower breathing rate than would occur without paced breathing therapy may aid in the effectiveness of inhalation therapy. For example, inducing slower breathing may assist in more complete delivery of a therapeutic substance released by the drug delivery component to the patient's lungs a (e.g., a slower breath may enable absorption of a greater amount of therapeutic substance by lung tissue) and/or may provide other benefits. In some implementations, utilizing the breathing circuit to induce a tidal volume different from the tidal volume without paced breathing therapy can also aid in the effectiveness of inhalation therapy. For example, inducing a greater tidal volume can assist in delivery of more of a therapeutic substance released by the drug delivery component to the patient's lungs (e.g., a “deeper breath” may enable more therapeutic substance to be absorbed by lung tissue) and/or may provide other benefits. In some implementations, utilizing the breathing circuit to induce more regular breathing may aid in the effectiveness of inhalation therapy. For example, inducing more regular breathing may assist the accuracy of delivery of a therapeutic substance released by the drug delivery portion to the patient (e.g., a regular, predictable breathing rate enables better calculation of how much substance to release) and/or may provide other benefits. In some implementations, utilizing the breathing circuit to control the ratio of inhalation duration to exhalation duration may aid in the effectiveness of inhalation therapy. For example, controlling the I/E ratio may assist in delivery of more of a therapeutic substance released by the drug delivery component to the patient's lungs (e.g., adjusting the I/E ratio to ensure that therapeutic substance is not exhaled) and/or may provide other benefits. In some implementations, the use of paced breathing therapy with inhalation therapy may minimize the release of potentially hazardous drugs into the surrounding environment (e.g., by ensuring that the correct amount of therapeutic substance is released by the drug delivery component and that a maximum amount of released therapeutic substance is absorbed by the lung tissue of the patient).

In some implementations, the signals from the control unit to the ventilator for controlling paced breathing may be used to determine the phase/state of the paced breathing cycle and thus control therapeutic substance delivery to the breathing circuit. For example, the signal from the control unit to the ventilator to create a high level pressure within the breathing circuit, thereby influencing the patient to inhale, may be used to trigger the drug delivery component to begin nebulization and delivery of a therapeutic substance to the breathing circuit or to begin the flow of a gaseous therapeutic substance to the breathing circuit. Similarly, a signal from the control unit to the ventilator to create a low level (i.e., lower than the high level) pressure in the breathing circuit, thereby influencing the patient to exhale, may be used to cease nebulization and delivery of a therapeutic substance to the breathing circuit or to cease delivery of a gaseous therapeutic substance to the breathing circuit.

In some implementations, signals from the control unit to the ventilator need not be directly used to trigger production/delivery of a compound to the patient or cessation thereof. For example, in some implementations, the ventilator may utilize its own separate integrated control unit for controlling paced breathing and may not readily be adapted to send signals to the drug delivery component. As such, in some implementations, the phase of the paced breathing cycle may be detected from the conditions (e.g., pressure/flow) within the breathing circuit itself and used to control drug delivery. As such, in some implementations, the breathing circuit may include one or more pneumatic coupling points in the paced breathing circuit to detect the state of the paced breathing cycle. In some implementations, the coupling point may include one or more sensors, transducers, and/or meters that measure pressure or flow through the breathing circuit in order to determine the pressure in the breathing circuit.

In some implementations, one or more electrical interfaces may be associated with the one or more pneumatic couplings to communicate activity in the breathing circuit to the control unit so that the phase/state of the paced breathing cycle can be monitored. For example, when the electrical interfaces communicate that the appropriate portion of the paced breathing cycle has been reached (e.g., inhalation or just before inhalation), the control unit may initiate the drug delivery portion to begin generation of a liquid or powder aerosol, to initiate opening the valve of a gas source, and/or to otherwise initiate delivery of a therapeutic substance to the breathing circuit. Similarly, detection of the appropriate portion of the paced breathing cycle (e.g., exhalation or just before exhalation) may initiate cessation of substance delivery to the breathing circuit.

In some implementations, as discussed above the control of breath tempo, tidal volume, regularity, I/E ratio, and/or other patient breath characteristics may be used by the systems and methods disclosed herein to improve administration of inhalation therapy. In some implementations, one or more characteristic thresholds may be set in order to trigger commencement and/or cessation of inhalation therapy. For example, one or more of a specific respiration tempo/rate, a specific tidal volume, a specific measure of respiration consistency/regularity, a specific inhalation/exhalation ratio, or other specific breath characteristic may be set as a trigger for the commencement of inhalation therapy/delivery of an inhaled therapeutic. As such, when the one or more thresholds for breath characteristics are set as a trigger for inhalation therapy, the breathing circuit may be used to administer paced breathing therapy to achieve breath characteristics matching the trigger characteristics. The control unit controlling the paced breathing therapy may be programmed so as to administer high and low pressure intervals to the patient such that the one or more trigger characteristics may be achieved. When the trigger characteristics are achieved (which may be determined by signals originating from the control unit of the paced breathing system or which may be sensed in the breathing circuit), inhalation therapy may be commenced (e.g., therapeutic substance generation may be tied to the inhalation cycle of the patient).

In some implementations, inhalation therapy may be ceased when the one or more of the breath characteristics of the patient do not match the trigger characteristics. In some implementations, the trigger characteristics may include a range of values, such that inhalation therapy may be ceased if one or more the breath characteristics of the patient fall outside (either higher or lower) the defined range. In some implementations, inhalation therapy may be ceased when a predetermined time period has elapsed, when a certain amount (e.g., by weight or volume) of therapeutic substance has been delivered to the patient, or may be ceased based on other factors.

As disclosed herein, the breathing circuit may be used to administer paced breathing therapy to a patient. In some implementations, for example, if it is determined that delivery of a therapeutic substance would benefit from one or more breath characteristics different from those of the paced breathing therapy, the breath characteristics of the patient may be altered through the use of different high/low pressures and/or high/low pressure durations from those of the paced breathing therapy being delivered to those that induce breath characteristics beneficial or desirable to the administration of inhalation therapy. Once the inhalation therapy is ceased (e.g., because the desired amount of therapeutic substance has been delivered, the prior paced breathing therapy may resume.

The invention may include a process for providing integrated paced breathing and inhalation therapy to a patient. The process may include an operation wherein paced breathing is established via a paced breathing circuit. As described herein, paced breathing may include monitoring the respiration of a patient to determine one or more the patient's breathing characteristics that are to be controlled (e.g., respiration frequency, tidal volume, respiration regularity, I/E ratio, and/or other characteristic of patient breathing/respiration). During the inhalation portion of the user's respiration a high pressure is supplied to the respiratory system of the patient. During the exhalation portion, a low pressure (relative to the high level) is supplied. One or more of the user's breathing characteristics are then compared to one or more target characteristics. One or more attributes of the high and/or low pressure provided to the patient by the breathing circuit (e.g., start time, end time, duration, pressure, etc.) may be adjusted in order to bring one or more of patient's breath characteristics towards the one or more target characteristics. For example, when the patient's breathing frequency is greater than a target frequency, the time over which the high pressure is supplied is increased, in a predetermined manner, and the time over which the low pressure is supplied is adjusted. Other characteristics of the high and/or low pressure supplied to the patient may be altered to change other breath characteristics. For example, in some implementations, the low pressure time may be adjusted in accordance with a fixed ratio between the high and low pressure times, or may be adjusted to coincide with the user's actual exhalation time. In this manner, the ratio between inhalation duration and exhalation duration (“I/E ratio”) may be controlled. The stage of the paced breathing provision is then determined. As described herein, in some implementations, this may include use of signals that meter provision of the high and low pressures from the breathing circuit to the patient. As such, these signals may be used to activate/deactivate provision of a therapeutic substance to the patient. In some implementations, this may include detecting the pressure within the breathing circuit to determine whether the high or low pressure is being provided (or is about to be provided) to the patient. Detection of the pressure in the breathing circuit may then be used to activate/deactivate provision of a therapeutic substance to the patient.

If it is determined that high pressure is being (or is about to be) provided to the patient, the therapeutic substance is then provided to the patient. As described herein, this may involve activating a drug delivery portion so that the therapeutic substance is delivered to the patient. This may involve, for example, activating a nebulizer so as to nebulize the therapeutic substance or opening a valve to release the therapeutic substance from a pressurized reservoir.

If it is determined that low pressure is being (or is about to be) provided to the patient, the therapeutic substance is either not provided to the patient or provision of the therapeutic substance to the patient is discontinued. Discontinuing provision of the therapeutic substance to the patient may include deactivating the drug delivery portion. This may involve, for example, discontinuing nebulization of the therapeutic substance or closing a valve gating a pressurized reservoir of the therapeutic substance.

The process may then monitor for any change that would affect the prior determination of the phase of the paced breathing cycle and thus affect whether therapeutic substance delivery should be commenced or discontinued.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

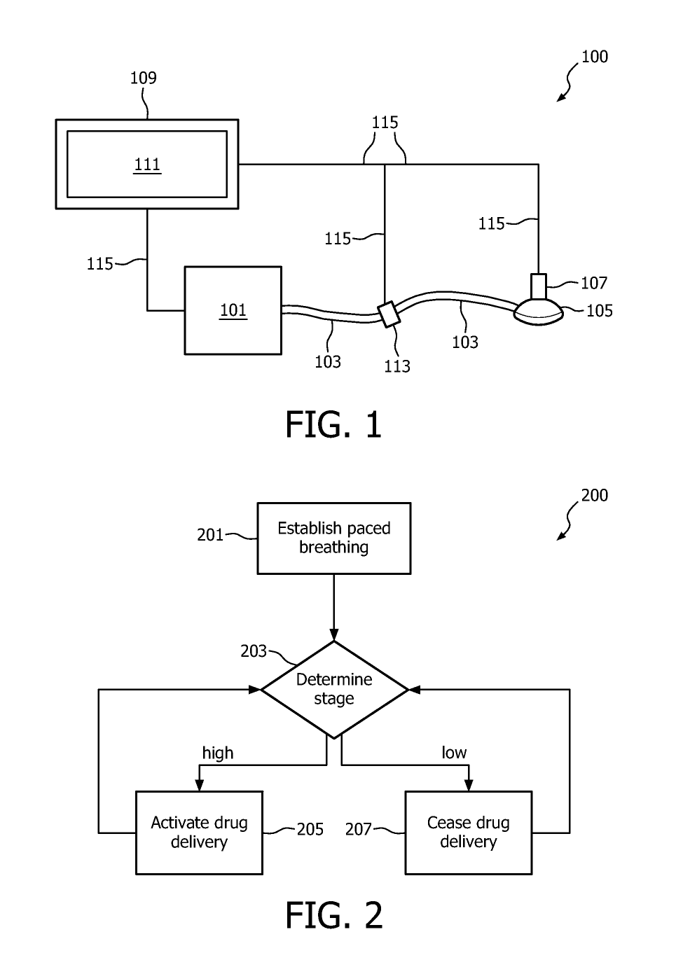

FIG. 1 is an example of a system for integrated paced breathing and inhalation therapy according to various implementations of the invention.

FIG. 2 is an example of a process for integrated paced breathing and inhalation therapy according to various implementations of the invention.

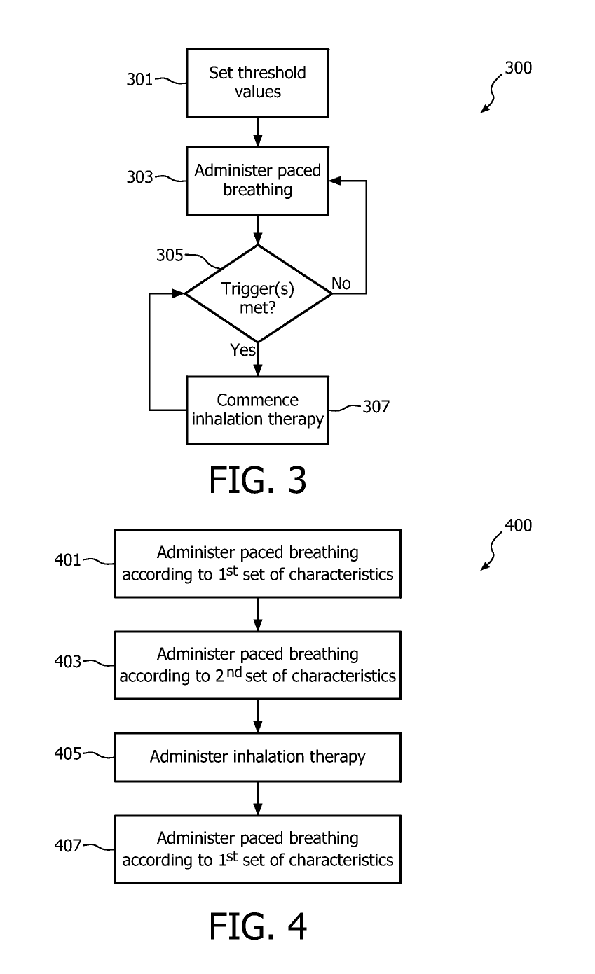

FIG. 3 is an example of a process for integrated paced breathing and inhalation therapy according to various implementations of the invention.

FIG. 4 is an example of a process for integrated paced breathing and inhalation therapy according to various implementations of the invention.

One aspect of the invention provides systems and methods for integrated ventilatory paced breathing and inhaled substance therapy. A system for combined ventilatory paced breathing and inhaled substance therapy may include a breathing circuit, a drug delivery component, and a control component. FIG. 1 illustrates a system 100, which is an example of a system for integrated paced breathing and inhalation therapy according to various aspects of the invention. System 100 includes breathing circuit that may include a ventilator 101 or other pressure manipulation device, a breathing conduit 103, and a patient interface 105. In some implementations, patient interface 105 may include a facemask (illustrated in FIG. 1), a nasal cannula, and/or other device that connects the patient to breathing conduit 103. In some implementations, breathing conduit 103 may be or include flexible tubing, various connections, and/or other elements that transmit gas between ventilator 101 and the respiratory system of the patient. The breathing circuit may be configured to selectively provide different airway pressures to the respiratory system of a patient and/or otherwise provide airflow or breathing regulation or assistance to the respiratory system of the patient.

In some implementations, the breathing circuit may enable control of one or more characteristics of patient breathing such as, for example, patient breathing rate (e.g., slowing down or speeding up the breathing rate from what it would be for the patient without the paced breathing therapy), breath volume/tidal volume (e.g., increasing or decreasing the tidal volume from what it would be for the patient without paced breathing therapy), ratio between inhalation duration and exhalation duration (“E/I ratio”), breath regularity (e.g., consistency of breathing rate, E/I ratio, or other characteristic over a period of time), and/or other breath characteristics. In some implementations, the breathing circuit may be used to provide positive end expiratory pressure (“PEEP”—e.g., to provide suitable pressure to keep certain alveoli open and thus ensure recruitment of unstable parts of the lung), to manipulate flow rates (the flow rate of therapeutic substance delivery, along with the particle size [if any] of the therapeutic substance, can be manipulated to deliver the therapeutic substance to specifically targeted portions of a patient's respiratory system), and/or to provide other benefits. The characteristics of these techniques (i.e., PEEP, flow rate manipulation, etc.) may be selectively controlled according to some implementations of the invention for the benefit of providing inhalation therapy.

System 100 may also include a drug delivery component 107 to deliver a drug, compound, or therapeutic substance to the patient. In some implementations, drug delivery component 107 may vary depending on the type of compound being delivered to the patient. For example, to administer an aerosolized, powder or liquid, drug delivery component 107 may include a nebulizer as a selective drug delivery portion of drug delivery component. The nebulizer may be or include a compression nebulizer, an ultrasonic nebulizer, a piezoelectric nebulizer, and/or other type of nebulizer. The nebulizer may include a reservoir of the therapeutic substance to be delivered to the patient, any conduits or element necessary to deliver the substance to be nebulized from the reservoir to the nebulizing portion of the nebulizer, any conduits or elements necessary to provide nebulized compound to the breathing circuit and/or respiratory system of the patient, any electrical or physical components necessary to actuate the nebulizer, and/or other elements. In some implementations the aerosolized substance may be introduced into the paced breathing circuit at mask 105 or other location of the breathing circuit (e.g., anywhere along breathing conduit 103) depending on the gravitational properties of the aerosol particles (size, density, etc.) and/or other factors.

To administer a gaseous therapeutic substance, drug delivery component 107 may include a compound source (e.g., a tank of compressed gas [e.g., oxygen or other gas] containing the compound to be delivered), a conduit to deliver the gas from the source to the breathing circuit, and a valve as a selective drug delivery portion to selectively allow flow from the source through the conduit to the breathing circuit and/or the respiratory system of the patient.

In some implementations wherein a therapeutic substance is aerosolized prior to delivery to the patient, the flow energy in the breathing circuit may be used to aerosolize and transport the drug/compound to the airway. In some implementations, the therapeutic substance may be aerosolized in a nebulizer located outside of the breathing circuit and introduced into the paced breathing circuit at mask 105 or other location depending on the gravitational properties of the aerosol particles (size, density, etc.) and/or other considerations. In instances wherein the therapeutic substance used for inhalation therapy is a gas, the gas may be introduced more flexibly into the breathing circuit.

System 100 may include a control component that may be or include control unit 109. Control unit 109 may include a computer-implemented device having one or more computer processors (e.g., microprocessors) and associated memory and/or data storage. In some implementations, the one or more processors may be configured to perform one or more operations relating to paced breathing and inhalation therapy described herein. Control unit 109 may include and the one or more processors may be configured by a control application 111. Although illustrated separately in FIG. 1, in some implementations, ventilator 101 may include control unit 109 and control application 111.

Control application 111 may include and/or be programmed with a machine-readable (e.g., processor-readable) paced breathing algorithm so as to provide various airway pressures to the respiratory system of the patient for treatment of one or more medical conditions. In some implementations, paced breathing therapy employs positive air pressure (PAP) cues as a more natural proprioceptive signal to coach patients to breathe at a slow, therapeutically significant breath rate (e.g., <10 breaths per minute (BPM)). This may provide several physiological benefits such as, for example, reduction of blood pressure and/or other benefits. As discussed herein, paced breathing may be used to control breathing rate, tidal volume, regularity of breathing, I/E ratio, and/or other breath characteristics. Paced breathing may also be used to provide positive end expiratory pressure (“PEEP”—e.g., to provide suitable pressure to keep certain alveoli open and thus ensure recruitment of unstable parts of the lung), to manipulate flow rates (the flow rate of therapeutic substance delivery, along with the particle size [if any] of the therapeutic substance, can be manipulated to deliver the therapeutic substance to specifically targeted portions of a patient's respiratory system), and/or to provide other benefits. The characteristics of these techniques (i.e., PEEP, flow rate manipulation, etc.) may be selectively controlled according to some implementations of the invention for the benefit of providing inhalation therapy. The paced breathing algorithm used to control airway pressure may vary according to the desired patient breath characteristics (and/or the characteristics of the therapy sought to be imposed upon the patient).

Paced breathing initially involves monitoring the respiration of a patient to determine the patient's breathing or respiration frequency. Respiration can be considered to comprise an inspiration (i.e., inhalation) portion or time and an expiration (i.e., exhalation) portion or time. During the inspiration portion of the user's respiration a HI pressure of the Bi-level PAP is supplied, and during the expiration portion, the LO pressure of the Bi-level PAP is supplied. Depending on the desired characteristics of patient respiration, one or more current characteristics of the patient's respiration may be monitored, compared to one or more target characteristics, and the pressure based breathing cues (i.e., the HI and LO pressures) may be adjusted accordingly.

For example, if breath frequency were to be controlled, the user's breathing frequency would be monitored and then compared to a target breathing frequency. When the user's breathing frequency is greater than the target frequency, the time over which the HI pressure of the Bi-level PAP is supplied is increased, in a predetermined manner, and the time over which the LO pressure of the Bi-level PAP is supplied is adjusted. The LO pressure time may be adjusted in accordance with a fixed ratio between the HI and LO pressure times, or may be adjusted to coincide with the user's actual expiration time. Those skilled in the art will appreciate that the LO pressure time may also comprise application of negative pressure.

Additional information relating to positive airway pressure and paced breathing therapy may be found in U.S. Pat. No. 6,105,575, and U.S. Pat. No. 7,556,038 entitled “System and Methods for Controlling Breathing Rate”), the disclosure of each of which is hereby incorporated by reference herein in their entirety.

The paced breathing algorithm may be implemented using one or more breathing signals from control unit 109 to ventilator 101 that instruct ventilator to create a specific level of positive pressure in the breathing circuit or otherwise cause flow within the breathing circuit. In some implementations, the breathing signal may also be used to control the timing of drug/therapeutic substance delivery to optimize therapy delivery and economize on the use of expensive compounds. In some instances, control of therapeutic substance delivery based on a paced breathing signal may increase the effectiveness of inhalation therapy by, for example, significantly enhancing the transport of inhaled substances to the lung.

For example, in some implementations utilizing the breathing circuit to induce a slower breathing rate than would occur without paced breathing therapy may aid in the effectiveness of inhalation therapy. For example, inducing slower breathing may assist in more complete delivery of a therapeutic substance released by the drug delivery component to the patient's lungs a (e.g., a slower breath may enable absorption of a greater amount of therapeutic substance by lung tissue) and/or may provide other benefits.

In some implementations, utilizing the breathing circuit to induce a tidal volume different from the tidal volume without paced breathing therapy can also aid in the effectiveness of inhalation therapy. For example, inducing a greater tidal volume can assist in delivery of more of a therapeutic substance released by the drug delivery component to the patient's lungs (e.g., a “deeper breath” may enable more therapeutic substance to be absorbed by lung tissue) and/or may provide other benefits.

In some implementations, utilizing the breathing circuit to induce more regular breathing may aid in the effectiveness of inhalation therapy. For example, inducing more regular breathing may assist the accuracy of delivery of a therapeutic substance released by the drug delivery portion to the patient (e.g., a regular, predictable breathing rate enables better calculation of how much substance to release) and/or may provide other benefits.

In some implementations, utilizing the breathing circuit to control the ratio of inhalation duration to exhalation duration may aid in the effectiveness of inhalation therapy. For example, controlling the I/E ratio may assist in delivery of more of a therapeutic substance released by the drug delivery component to the patient's lungs (e.g., adjusting the I/E ratio to ensure that therapeutic substance is not exhaled) and/or may provide other benefits.

In some implementations, utilizing the breathing circuit to provide positive end expiratory pressure (PEEP) treatment may aid in the effectiveness of inhalation therapy. For example, providing PEEP treatment may enable recruitment of unstable portions of the lung by providing sufficient positive pressure to keep certain alveoli open within the lung of a patient. Use of these unstable portions may enable more of a therapeutic substance to be provided to the patient. Furthermore, recruitment of unstable portions of the lung may enable delivery of therapeutic substances to those previously inaccessible portions of the lung. As such, the pressure within the respiratory system of a patient may be a characteristic that is varied/manipulated according to various implementations of the invention.

In some implementations, utilizing the breathing circuit to control flow rates within the respiratory system of the patient may aid in the effectiveness of inhalation therapy. For example, manipulation of the flow rate within the respiratory system of the patient, along with the specific size of the particle of the therapeutic substance to be delivered (or other characteristic of the therapeutic substance to be delivered) may enable targeted delivery of the therapeutic substance to specific portions of the respiratory system of the patient (e.g., delivery of larger particles deeper into the lung) and may therefore provide more effective delivery of a therapeutic substance. Thus, the flow rate within the respiratory system of the patient may be a characteristic that may be varied/manipulated according to various implementations of the invention.

In some implementations, the use of paced breathing therapy with inhalation therapy may minimize the release of potentially hazardous drugs into the surrounding environment (e.g., by ensuring that the correct amount of therapeutic substance is released by the drug delivery component and that a maximum amount of released therapeutic is absorbed by the lung tissue of the patient). For example, for systems used in the home, it may be desirous to minimize the introduction of drugs or therapeutic substances into the surrounding environment so as to minimize exposure by family members of the patient. Similarly minimizing the introduction of drugs/therapeutic substances into the surrounding environment in a clinical setting (e.g., hospital, nursing home, etc.) may be desirable so as to minimize exposure by medical professionals. Use of the paced breathing signal to control drug delivery ensures that more of a therapeutic substance is delivered to the patient's system. As such, less of the compound accidentally escapes from the system into the surrounding environment.

In some implementations, the signals from control unit 109 to ventilator 101 for controlling paced breathing may be used to determine the phase/state of the paced breathing cycle and thus control therapeutic substance delivery to the breathing circuit. For example, the signal from control unit 109 to ventilator 101 to create a high level pressure within the breathing circuit, thereby influencing the patient to inhale, may be used to trigger drug delivery component 107 (e.g., via a communications link 115) to begin nebulization and delivery of a therapeutic substance to the breathing circuit or to begin the flow of a gaseous therapeutic substance to the breathing circuit. Similarly, a signal from control unit 109 to ventilator 101 to create a low level (i.e., lower than the high level) pressure in the breathing circuit, thereby influencing the patient to exhale, may be used to cease nebulization and delivery of a therapeutic substance to the breathing circuit or to cease delivery of a gaseous therapeutic substance to the breathing circuit.

In some implementations, the timing of delivery and cessation of a therapeutic substance to the breathing circuit may be based on a determination of the appropriate time to introduce the substance into the patient's respiratory system and thus may not directly coincide with the beginning and end of inhalation. For example, the therapeutic substance may be generated and/or delivered into the breathing circuit prior to the beginning of actual inhalation by the patient so as to account for time necessary for the substance to travel through the breathing circuit into the appropriate point in the patient's respiratory system. Similarly, cessation of therapeutic substance production and/or delivery may occur prior to the cessation of inhalation by the patient so that all produced/delivered substance reaches the patients respiratory system.

In some implementations, signals from control unit 109 to ventilator 101 need not be directly used to trigger production/delivery of a therapeutic substance to the patient or cessation thereof. For example, in some implementations, ventilator 109 may utilize its own separate control application for controlling paced breathing and/or may not readily be adapted to send signals to drug delivery component 107. As such, in some implementations, the phase of the paced breathing cycle may be detected from the conditions/flow within the breathing circuit itself and used to control drug delivery

In some implementations, the breathing circuit may include one or more pneumatic coupling points in the paced breathing circuit to detect the state paced breathing cycle. FIG. 1 illustrates coupling point 113 integrated into breathing conduit 103. In some implementations, several coupling points may used or may be used at different points in the breathing circuit. In some implementations, coupling point 113 may include one or more sensors, transducers, and/or meters (e.g., pressure transducers, flow meters, etc.) that measure pressure or flow rate through the breathing circuit (or conduit 103) in order to determine the phase/stage of paced breathing therapy. In some implementations, a plurality of different breathing circuits may be provided, each having one of a plurality of different types of pneumatic coupling.

In some implementations, one or more electrical interfaces 115 may be associated with the one or more pneumatic couplings 113 to communicate activity in the breathing circuit to control unit 109 so that the phase/state of the paced breathing cycle can be monitored. In some implementations, electrical interfaces 115 may include any suitable communications link such as, for example, a copper wire, a fiber optic cable, or other communications link. For example, when electrical interface 115 communicates that the appropriate portion of the paced breathing cycle has been reached (e.g., inhalation or just before inhalation), control unit 109 may initiate a drug delivery portion 107 to begin generation of a liquid or powder aerosol, to initiate opening the valve of a gas source, and/or to otherwise initiate delivery of a therapeutic substance to the breathing circuit. Similarly, detection of the appropriate portion of the paced breathing cycle (e.g., exhalation or just before exhalation) may initiate cessation of substance delivery to the breathing circuit.

FIG. 2 illustrates a process 200 for providing integrated paced breathing and inhalation therapy to a patient. In an operation 201, paced breathing is established via a paced breathing system, such as system 100. As described herein, paced breathing may include monitoring the respiration of a patient to determine the current state of one or more characteristics of the patient's breathing or respiration that are to be controlled (e.g., respiration frequency, tidal volume, respiration regularity, I/E ratio, and/or other characteristic of patient respiration). During the inhalation portion of the user's respiration a high pressure is supplied to the respiratory system of the patient. During the exhalation portion, a low pressure (relative to the high level) is supplied. One or more of the user's breathing characteristics are then compared to one or more target characteristics. One or more attributes of the high and/or low pressure provided to the patient by the breathing circuit (e.g., start time, end time, duration, pressure, etc.) may be adjusted in order to bring one or more of the patient's breath characteristics towards the one or more target characteristics. For example, when the patient's breathing frequency is greater than the target frequency, the time over which the high pressure is supplied is increased, in a predetermined manner, and the time over which the low pressure is supplied is adjusted. The low pressure time may be adjusted in accordance with a fixed ratio between the high and low pressure times, or may be adjusted to coincide with the user's actual exhalation time. Other characteristics of the high and/or low pressure supplied to the patient may be altered to change other breath characteristics.

In an operation 203, a stage of the paced breathing provision is determined. As described herein, in some implementations, this may include use of signals from control unit 109 to ventilator 101 that meter provision of the high and low pressures from the breathing circuit to the patient. As such, these signals may be used to activate/deactivate provision of a therapeutic substance to the patient. In some implementations, this may include detecting the pressure within the breathing circuit (e.g., via pneumatic coupling 113) to determine whether the high or low pressure is being provided (or is about to be provided) to the patient. Detection of the pressure in the breathing circuit may then be used to activate/deactivate provision of a therapeutic substance to the patient.

If it is determined (e.g., by control unit 109/pneumatic coupling 113) that high pressure is being (or is about to be) provided to the patient, process 200 proceeds to an operation 205, wherein the therapeutic substance is provided to the patient. As described herein, this may involve activating a drug delivery portion (e.g., drug delivery component 107) so that the therapeutic substance is delivered to the patient. This may involve, for example, activating a nebulizer of the drug delivery portion so as to nebulize the therapeutic substance or opening a valve to release the therapeutic substance from a pressurized reservoir.

If it is determined (e.g., by control unit 109/pneumatic coupling 113) that low pressure is being (or is about to be) provided to the patient, process 200 proceeds to an operation 207, wherein the therapeutic substance is either not provided to the patient or wherein provision of the therapeutic substance to the patient at the drug delivery portion is discontinued. Discontinuing provision of the therapeutic substance to the patient may include deactivating the drug delivery portion. This may involve, for example, discontinuing nebulization of the therapeutic substance or closing a valve gating a pressurized reservoir of the therapeutic substance.

After either operation 205 or 207, process 200 may then return to operation 203 wherein the paced breathing cycle is monitored for any change that would affect the determination of operation 203 and thus affect whether therapeutic substance delivery should be commenced or discontinued.

In some implementations, as discussed above the control of breath tempo, tidal volume, regularity, I/E ratio, flow rates, and/or other patient breath characteristics may be used by the systems and methods disclosed herein to improve administration of inhalation therapy. As such, the invention may include a process such as, for example, process 300, which is an example of a process for utilizing specific values of one or more breath characteristics controlled by paced breathing as triggers for initiation of inhalation therapy. Process 300 may include an operation 301, wherein one or more breath characteristic threshold values may be set. These values may serve as triggers for the commencement and/or cessation of an inhalation therapy session (while the commencement and cessation of provision of therapeutic substance to the patient may be gated by the phase of the breathing cycle of the patient such as disclosed in process 200). Examples of value may include a specific respiration tempo/rate (e.g., breaths per minute), a specific tidal volume (e.g., in milliliters), a specific measure of respiration consistency/regularity (e.g., standard deviation of breath rate over time), a specific inhalation/exhalation ratio, a specific pressure within the respiratory system of the patient, a specific flow rate within the respiratory system of the patient, or other specific breath characteristic. In an operation 303, paced breathing therapy may be commenced, with a paced breathing algorithm focused on bringing the breath characteristics of the patient to the trigger characteristics. In an operation 305, it may be determined whether the one or more trigger characteristic values match actual patient breathing characteristics (e.g., by measuring flow through the breathing circuit by pneumatic couplings 113). If the trigger(s) is/are met, inhalation therapy may be commenced in an operation 307. The inhalation therapy may be administered commensurate with the patient's breath cycle such as, for example, as described in process 200. If the trigger(s) is/are not met, process 300 may return to operation 303, wherein paced breathing therapy is administered with the goal of bringing the patients breathing characteristics in line with the trigger(s).

In some implementations, the inhalation therapy session may be ceased when the one or more of the breath characteristics of the patient do not match the trigger characteristics. For example, patient breath characteristics may deviate from the trigger characteristics without a change in the administration of the paced breathing therapy (e.g., the patient may consciously or unconsciously “resist” the paced breathing therapy). In some implementations, process 300 may continuously monitor the breath characteristics so that inhalation therapy ceases when the actual breath characteristics of the patient deviate from the trigger characteristics and return to the administration of inhalation therapy when the patient breath characteristics return to those that match the trigger(s) (e.g., process 300 may return to decision 305 continuously or until the inhalation therapy “session” is completed).

In some implementations, the trigger characteristics may include a range of values, such that inhalation therapy may be ceased if one or more the breath characteristics of the patient fall outside (either higher or lower) the defined range. In some implementations, inhalation therapy may be ceased when a predetermined time period has elapsed (e.g., an inhalation therapy “session” has completed), when a certain amount (e.g., by weight or volume) of therapeutic substance has been delivered to the patient (e.g., an inhalation therapy “session” has completed), or may be ceased based on other factors.

As disclosed herein, the breathing circuit may be used to administer paced breathing therapy to a patient. In some implementations, for example, if it is determined that delivery of a therapeutic substance would benefit from one or more breath characteristics different from those of the paced breathing therapy, the breath characteristics of the patient may be altered through the use of different high/low pressures and/or high/low pressure durations from those of the paced breathing therapy being delivered to those that induce breath characteristics beneficial or desirable to the administration of inhalation therapy. Once the inhalation therapy is ceased (e.g., because the desired amount of therapeutic substance has been delivered, the prior paced breathing therapy may resume.

FIG. 4 illustrates a process 400, which is an example of a process for utilizing control provided by paced breathing therapy to administer inhalation therapy, wherein one or more breath characteristics desired for inhalation therapy are different from one or more breath characteristics desired for paced breathing therapy. Process 400 includes an operation 401, wherein paced breathing therapy is administered to a patient (e.g., via the breathing circuit of system 100), the paced breathing therapy influencing one or more of the patient's breathing characteristics (e.g., rate, tidal volume, I/E ratio, regulatory, flow rates, and/or other characteristic) towards a first set of one or more target breathing characteristics. The first set of target breathing characteristics may be selected for the purpose of providing therapeutic paced breathing benefits to the patient.

In an operation 403, the breathing circuit influences one or more breathing characteristics of the patient towards a second set of one or more target breathing characteristics, wherein a value/metric of at least one breathing characteristic of the second set of target breathing characteristics is different from a value/metric of at least one breathing characteristic of the first set of one or more target breathing characteristics. In some implementations, the second set of one or more breathing characteristics are selected for the purpose of delivering a therapeutic substance to the respiratory system of the patient (e.g., a rate, tidal volume, I/E ratio, regulatory, flow rates, or other characteristic that maximizes effectiveness of delivery of the therapeutic substance). In an operation 405, an inhalation therapy session is commenced (e.g., inhalation therapy is administered to the patient according to process 200).

When the inhalation therapy session is completed (e.g., a predetermined time period has passed, a predetermined number of respiratory cycles have occurred, a predetermined amount of therapeutic substance has been administered, or other metric), paced breathing is then administered to the patient according to the first set of target breathing characteristics in an operation 407.

In some implementations, a tangible computer readable medium having computer readable instructions thereon that can configure one or more computer processors to perform some or all of the features and functions of the method described herein can be provided. The systems and methods described herein are provided as examples only. Those having skill in the art will appreciate that the invention described herein may work with various system configurations and that other order of operations may exist for the processes/methods described herein. Accordingly, more or less of the aforementioned system components may be used and/or combined in various embodiments. Additionally, additional operations for methods may be performed while others may be omitted and/or operations may be performed in different orders.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A system for providing integrated paced breathing and inhalation therapy to a patient, comprising:
a breathing circuit including a patient interface portion, a breathing conduit, and a pressure manipulation portion, wherein the breathing circuit alternately supplies a high and a low airway pressure from the pressure manipulation portion to a respiratory system of the patient via the patient interface portion, wherein the high airway pressure coincides with inhalation by the patient, and wherein the low airway pressure coincides with exhalation by the patient; and
a drug delivery portion including a reservoir that stores a therapeutic substance and a selective delivery portion, wherein the selective delivery portion is activated so as to deliver at least a portion of the therapeutic substance from the reservoir to the respiratory system of the patient during inhalation by the patient, and wherein the selective delivery portion is deactivated so as to cease delivery of the therapeutic substance from the reservoir to the respiratory system of the patient during exhalation by the patient.

2. The system of claim 1, wherein one or more of the high or low airway pressures are supplied to the patient so as to bring at least one breath characteristic of the patient to a predetermined trigger value, and wherein the selective delivery portion is activated so as to deliver at least a portion of the therapeutic substance from the reservoir to the respiratory system of the patient during inhalation by the patient only when the at least one breath characteristic of the patient equals the trigger value.

3. The system of claim 2, wherein the at least one breath characteristic is one or more of a respiration rate, a tidal volume, a ratio of inhalation duration to exhalation duration, or a measure of respiratory regularity.

4. The system of claim 1, wherein one or more of the high or low airway pressures are supplied to the patient so as to bring at least one breath characteristic of the patient to one of a first and a second target value, wherein the selective delivery portion is activated so as to deliver at least a portion of the therapeutic substance from the reservoir to the respiratory system of the patient only when the at least one breath characteristic equals one of the first or the second target value.

5. The system of claim 1, wherein the breathing circuit supplies the high pressure to the respiratory system of the patient in response to a high pressure signal, wherein the breathing circuit supplies low pressure to the respiratory system of the patient in response to a low pressure signal, and wherein the selective delivery portion is activated by the high pressure signal and deactivated by the low pressure signal.

6. The system of claim 5, further comprising a control unit that sends the high pressure signal and the low pressure signal to the pressure manipulation portion.

7. The system of claim 6, wherein the control unit sends the high pressure signal and the low pressure signal to the drug delivery component.

8. The system of claim 1, further comprising a sensor located on the breathing circuit that senses delivery of high and low pressure to the respiratory system of the patient, wherein the sensor activates the drug delivery portion when delivery of high pressure is sensed and deactivates the drug delivery portion when low pressure is sensed.

9. The system of claim 1, wherein the selective delivery portion includes a nebulizer, and wherein activation of the selective delivery portion includes nebulization of the at least a portion of the therapeutic substance.

10. The system of claim 1, wherein the therapeutic substance is a liquid or a powder.

11. The system of claim 1, wherein the reservoir includes a pressurized gas source and wherein the selective delivery portion includes a valve that when activated, delivers gas to the respiratory system of the patient.

12. The system of claim 1, wherein the patient interface portion includes a mask.

13. A method for providing integrated paced breathing and inhalation therapy to a patient, comprising:
alternately providing a high and a low airway pressure to a respiratory system of the patient using a breathing circuit that includes a patient interface portion, a breathing conduit, and a pressure manipulation portion, wherein the high airway pressure coincides with inhalation by the patient and the low airway pressure coincides with exhalation by the patient; and
delivering a therapeutic substance to the respiratory system of the patient during the provision of the high airway pressure to the respiratory system of the patient, wherein delivery of the therapeutic substance to the respiratory system of the patient is discontinued during provision of the low airway pressure to the respiratory system of the patient.

14. The method of claim 13, wherein one or more of the high or low airway pressures are provided to the patient so as to bring at least one breath characteristic of the patient to a predetermined trigger value, and wherein at least a portion of the therapeutic substance is delivered from the reservoir to the respiratory system of the patient during provision of the high airway pressure to the respiratory system of the patient only when the at least one breath characteristic of the patient equals the trigger value.

15. The method of claim 14, wherein the at least one breath characteristic is one or more of a respiration rate, a tidal volume, a ratio of inhalation duration to exhalation duration, or a measure of respiratory regularity.

16. The method of claim 13, wherein one or more of the high or low airway pressures are provided to the patient so as to bring at least one breath characteristic of the patient to one of a first and a second target value, wherein the therapeutic substance is delivered from the reservoir to the respiratory system of the patient during provision of the high airway pressure to the respiratory system of the patient only when the at least one breath characteristic equals one of the first or the second target value.

17. The method of claim 13, wherein high airway pressure is supplied to the respiratory system of the patient by the breathing circuit in response to a high pressure signal, wherein low pressure is supplied to the respiratory system of the patient by the breathing circuit in response to a low pressure signal, and wherein delivery of the compound is triggered by the high pressure signal and discontinued by the low pressure signal.

18. The method of claim 17, wherein the high pressure signal and the low pressure signal are sent to the pressure manipulation portion by a control unit.

19. The method of claim 18, wherein the control unit sends the high pressure signal and the low pressure signal to the drug delivery component.

20. The method of claim 13, wherein the breathing circuit includes a sensor that senses delivery of high and low pressure delivery to the respiratory system of the patient, wherein the sensor instructs a drug delivery portion to deliver the therapeutic substance to the respiratory system of the patient when delivery of high pressure is sensed, and wherein the sensor instructs the drug delivery portion to deactivates delivery of the therapeutic substance to the respiratory system of the patient when low pressure is sensed.

21. The method of claim 13, wherein delivery of the therapeutic substance to the respiratory system of the patient uses a nebulizer, and wherein the therapeutic substance is nebulized during delivery of the therapeutic substance to the respiratory system of the patient.

22. The method of claim 13, wherein the therapeutic substance is a liquid or a powder.

23. The method of claim 13, wherein the therapeutic substance comprises a gas, and wherein delivery of the therapeutic substance includes activating a valve associated with a pressurized source of the gas, thereby delivering the gas from the pressurized source to the respiratory system of the patient.

24. The method of claim 13, wherein the patient interface portion includes a mask.
